(19) United States
(12) Patent Application Publication
(10) Pub. No.: US 2013/0138063 A1
(43) Pub. Date: May 30, 2013

(54) ADHESIVE MEMBER

(71) Applicants: Neil Wiltshire, West Sussex (GB); Eric Davison, West Sussex (GB); Richard Darwood, West Sussex (GB)

(72) Inventors: Neil Wiltshire, West Sussex (GB); Eric Davison, West Sussex (GB); Richard Darwood, West Sussex (GB)

(73) Assignee: SALTS HEALTHCARE LIMITED, West Sussex (GB)

(21) Appl. No.: 13/497,714

(22) PCT Filed: Sep. 20, 2010
(86) PCT No.: PCT/GB10/51567

§ 371 Date: Feb. 14, 2013

(30) Foreign Application Priority Data
Sep. 22, 2009 (GB) 0916581.2

(51) Int. Cl. A61F 5/443

(52) U.S. Cl. CPC A61F 5/443
USPC 604/344

(57) ABSTRACT

An adhesive member (10) for adhering to and for covering a portion of a user's skin having an adhesive surface on at least one side thereof characterised in that the member includes a plurality of openings (12), each of which extends inwardly from an periphery of the member (10).

### DESCRIPTION OF INVENTION

This invention relates to an adhesive member for adhering to and for covering a portion of a user's skin. The adhesive member is generally planar and has an adhesive surface on at least one side thereof.

Such adhesive members can be used as dressings for covering a wound or may be used as part of an ostomy appliance for adhering the ostomy appliance to a user's skin. In the latter example, the adhesive member (often referred to as a “wafer”) is manufactured from a material which does not, usually, irritate the user's skin (e.g. hydrocolloid) and is generally circular, elliptical or oval. The adhesive member has an aperture in a central region thereof to receive a user's stoma. The aperture may be sized for a specific size of stoma. However, it is usually possible for the user to cut out a larger aperture in the adhesive member, if desired.

In a one-piece ostomy appliance, the adhesive member is connected directly to a bag (to receive waste). The user aligns the aperture in the adhesive member with their stoma and then moves the adhesive member towards and in contact with their skin. The user then presses the adhesive member against their skin to ensure that the member has adhered sufficiently to hold the appliance in situ.

In a two-piece ostomy appliance, the adhesive member is connected to a first connection part of the appliance, and is adhered to the user's skin in the same way as described above. The bag, however, is connected to a second connection part, which is removably connectable to the first connection part. The two-piece arrangement permits the user to remove the bag to clean it, or to replace it with a fresh bag, without having to remove the adhesive member from their skin. This results in reduced irritation to the user's skin underneath the adhesive member.

A problem with prior art adhesive members is that, due to the inherent irregular contours of a user's body, it is often the case that the adhesive member creases or ‘nicks up’ when installed. This can lead to leakage of waste fluids along these ‘creases’ from the central aperture which received the stoma and can also render the adhesive member (more) uncomfortable to the user. In addition, creases in the adhesive member can make the member more difficult to remove and can cause discomfort to the user. Creases and nicks in the adhesive member can also result in edges of the adhesive member lifting during use, which can lead to the adhesive member becoming disconnected from the user.

It is therefore an object of the present invention to provide an adhesive member which addresses this problem.

According to the first aspect of the invention, we provide an adhesive member for adhering to and for covering a portion of a user's skin having an adhesive surface on at least one side thereof characterised in that the member includes a plurality of openings, each opening extending inwardly from an periphery of the adhesive member.

Each opening may be generally elongate. Each opening may have a pair of opposing side walls.

The opposing side walls may be aligned substantially parallel with each other.

The opposing side walls may extend towards each other as they extend inwardly. For example, the opening may taper as it extends inwardly.

Each opening may extend inwardly towards a central region of the adhesive member in a direction which is substantially normal to a tangent of the periphery of the member adjacent an entrance to the opening.

The member may be substantially circular, elliptical or oval, and each opening may extend substantially radially inwardly towards the central region of the member.

At least one of the openings may be positioned substantially opposite another of the openings.

Each opening may be positioned substantially opposite another opening.

The openings may be aligned substantially parallel with each other.

At least one of the openings may be positioned substantially diametrically opposite another of the openings.

Each opening may be positioned substantially diametrically opposite another opening.

The opposing side walls of each opening may be moveable towards each other.

The member may include an aperture in the central region thereof and each opening may extend towards the aperture.

One half of the member may have more of said openings than the other half of the member.

One half of the member may have three of said openings. One half of the member may have two of said openings.

According to a second aspect of the invention, we provide an ostomy appliance including an adhesive member according to the first aspect of the invention.

According to a third aspect of the invention, we provide a one-piece ostomy appliance including an adhesive member according to the first aspect of the invention.

According to a fourth aspect of the invention, we provide a two-piece ostomy appliance including an adhesive member according to the first aspect of the invention.

### BRIEF DESCRIPTION OF DRAWINGS

Embodiments of the present invention will now be described by way of example only with reference to the accompanying drawings, of which:-

FIG. 1 is a plan view of an ostomy appliance including an adhesive member according to the present invention;

FIG. 2 is a plan view of a second embodiment of adhesive member according to the present invention;

FIG. 3 is a plan view of a third embodiment of adhesive member according to the present invention;

FIG. 4 is a plan view of a fourth embodiment of adhesive member according to the present invention;

FIG. 5 is a side view of the adhesive member of FIG. 4 in use; and

FIGS. 6 to 12 are plan views of further embodiments of adhesive member in accordance with the present invention.

### DETAILED DESCRIPTION

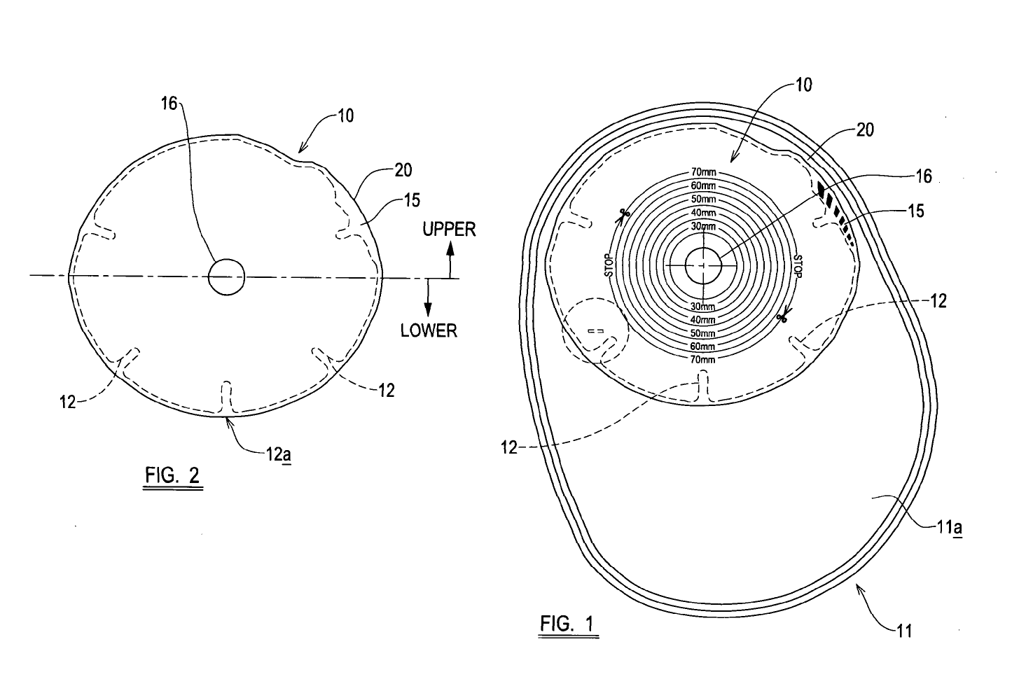

Referring to FIG. 1, this shows an ostomy appliance 11 including first embodiment of an adhesive member 10 in accordance with the present invention. The general construction of the ostomy appliance 11 is well known in the art and thus will not be discussed in detail here, except to state that it includes a bag 11a for receiving waste which is connected to the adhesive member 10 by known means.

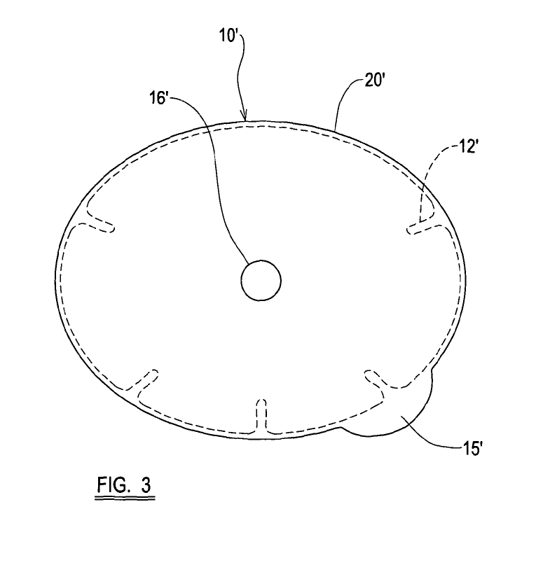

The adhesive member 10 (which is shown separately in FIG. 2) is generally planar, is generally circular in plan view and has an opening 16 to receive a user's stoma. The opening 16 can be increased in size, manually by a user, depending on the size (e.g. diameter) of their stoma. The purpose of the adhesive member 10 is to fix the ostomy appliance 11 to a user by adhering to the user's skin around their stoma, providing a fluid-tight seal. Thus, the side of the adhesive member 10 which faces the user has an adhesive surface. This side of the adhesive member 10 is covered by a covering 20 which is removed prior to use. The covering 20 has a graspable area/tab 15 to assist in its removal. Preferably, and as is well known in the art, the adhesive member 10 may be a hydrocolloid material, although any suitable material will suffice.

The adhesive member 10 of the present invention beneficially includes a plurality of openings 12, five in this example, each of which extends from a periphery of the member 10 towards a central region of the member 10. In this example, the openings 12 are each elongate and have a pair of opposing side walls 13, 14 which are aligned substantially parallel to each other. As an alternative, the openings 12 may taper, e.g. the walls 13, 14 may extend towards each other as they extend towards the central region, e.g. the aperture 16, of the member 10.

The openings 12 each extend towards the central region of the member 10 in a direction which is substantially normal to a tangent of the periphery of the member 10 adjacent an entrance 12a to each opening 12. In essence, this means that where the adhesive member 10 is generally circular, each opening 12 extends substantially radially inwardly towards the central region of the member 10.

In the present example, three openings 12 are provided in the lower half of the member 10 and two openings 12 are provided in the upper half of the member 10. The openings 12 provided in the upper half of the member 10 are positioned substantially opposite (e.g. diametrically opposite) one of the openings 12 provided in the lower half of the member 10. The remaining opening 12 is provided generally at the middle of the lower half of the member 12. Providing more openings 12 in the lower half (than the upper half) of the member 10 is beneficial, because this is the area where, generally, more creasing of the prior art adhesive members occurs. In addition, providing fewer openings 12 in the upper half is beneficial because the upper half takes more of the load from the ostomy appliance 11 (particularly when the bag is full of waste) and thus it is beneficial to have a greater area of adhesive in the upper half of the member 10. Of course, fewer or more openings 12 could be provided, and their positions around the periphery of the member 10 may be changed, as desired.

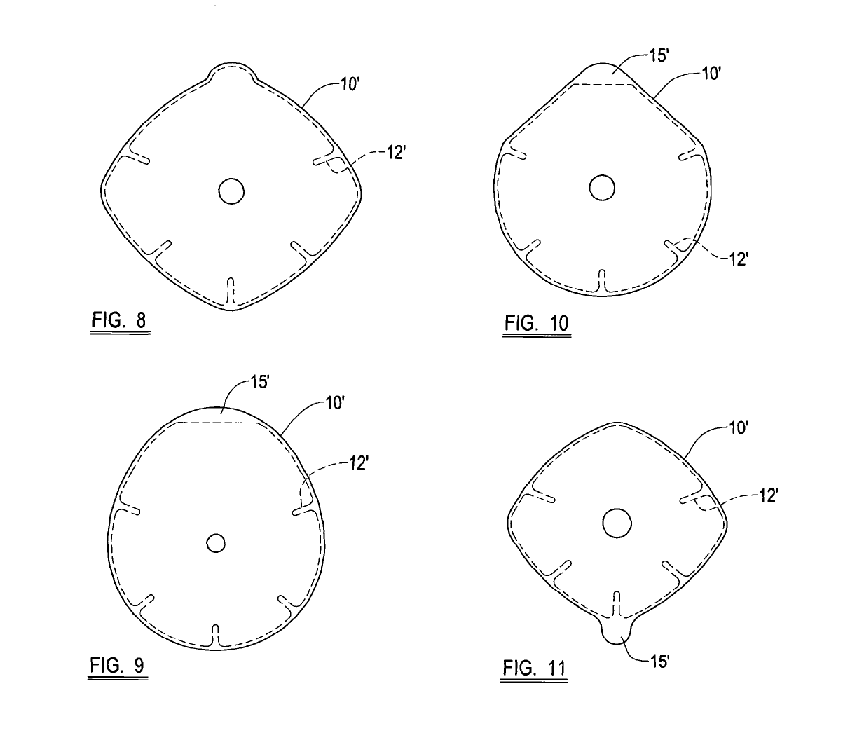

FIG. 5 illustrates how the openings 12 operate when the adhesive member is applied to a outwardly bulging surface of a user's skin. The side walls 13, 14 of the opening 12 move towards each other which ensures that no or little creasing occurs. This results in improved adhesion of the adhesive member 10 to the user's skin and decreases the likelihood of leakage from the stoma behind the adhesive member 10.

Generally, the user's skin around their stoma will bulge outwardly, and in this configuration, the side walls 13, 14 of the opening 12 will tend to move towards each other when the adhesive member 10 is applied to the user's skin. However, if the user's skin is concave around the stoma, the openings 12 will move away from each other as the adhesive member 10 is adhered to the user's skin. This will result in less stretching of the adhesive member 10 at its periphery which will improve adhesion and comfort to the user.

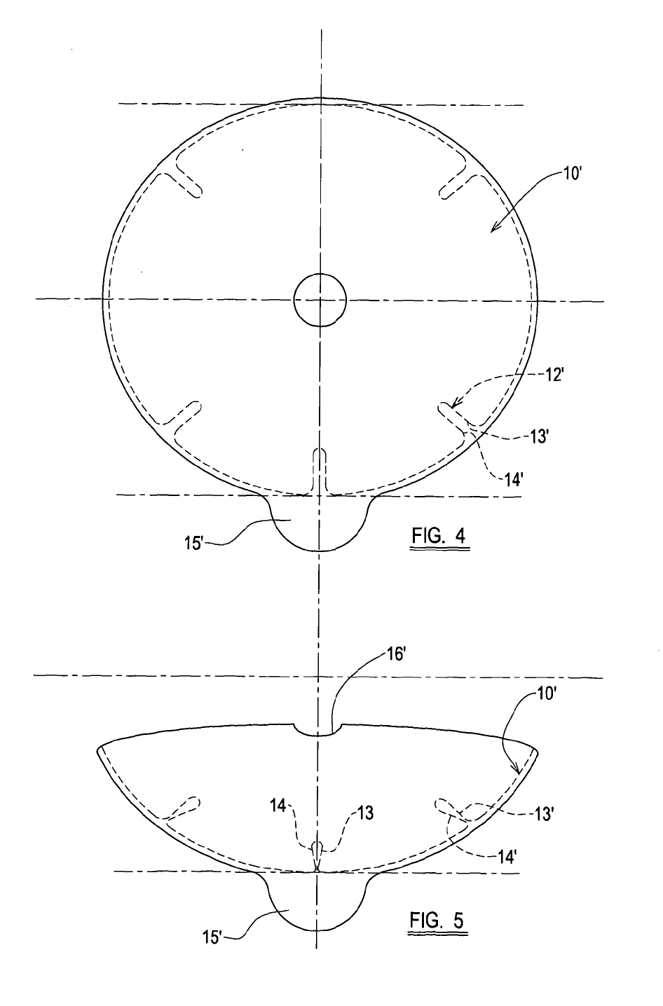

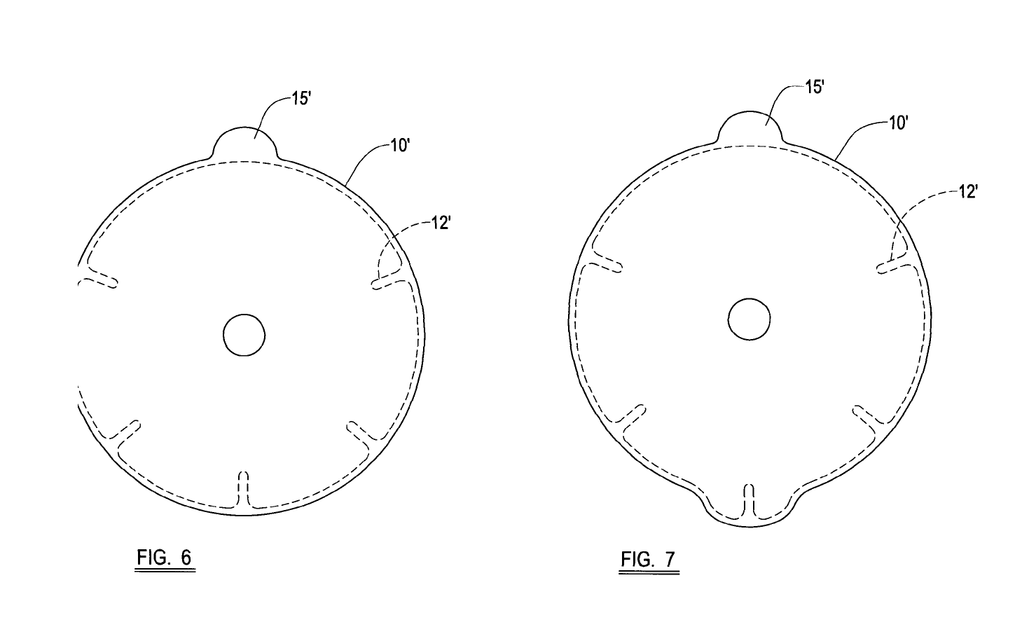

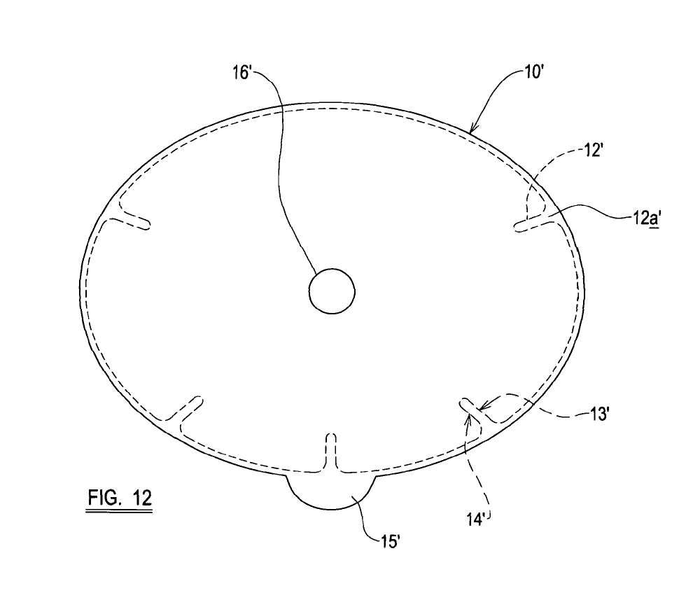

FIGS. 3 to 12 shows different embodiments of adhesive member 10′ in accordance with the present invention. Like features have been given the same reference numeral as for the adhesive member 10, but with the addition of a prime symbol (′). The embodiments differ from the adhesive member 10 in their peripheral shape. Some are circular, elliptical or oval, some are generally rectangular or diamond-shaped, and some are combinations of these shapes. It should be appreciated that any desired shape could be used, and the number of openings 12′ could be increased or decreased and their peripheral locations altered. These alternative shapes are therefore included as examples of how the openings 12, 12′ of the present invention can be utilised.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. An adhesive member (10) for adhering to and for covering a portion of a user's skin having an adhesive surface on at least one side thereof characterised in that the member includes a plurality of openings (12), each of which extends inwardly from an periphery of the member (10).

2. An adhesive member (10) according to claim 1 wherein each opening is generally elongate.

3. An adhesive member (10) according to claim 1 or claim 2 wherein each opening has a pair of opposing side walls (13, 14).

4. An adhesive member (10) according to claim 3 wherein the opposing side walls (13, 14) are aligned substantially parallel with each other.

5. An adhesive member (10) according to claim 3 wherein the opposing side walls extend towards each other as they extend inwardly.

6. An adhesive member (10) according to claim 1 wherein each opening (12) extends inwardly towards a central region of the adhesive member in a direction which is substantially normal to a tangent of the periphery of the member (10) adjacent an entrance (12a) to the opening.

7. An adhesive member (10) according to claim 1 wherein the member (10) is substantially circular, elliptical or oval, and each opening extends substantially radially inwardly towards the central region of the member (10).

8. An adhesive member (10) according to claim 1 wherein at least one of the openings (12) is positioned substantially opposite another of the openings (12).

9. An adhesive member (10) according to claim 1 wherein each opening (12) is positioned substantially opposite another opening (12).

10. An adhesive member (10) according to claim 9 wherein the openings (12) are aligned substantially parallel with each other.

11. An adhesive member (10) according to any one of claim 1 wherein at least one of the openings (12) is positioned substantially diametrically opposite (12) another of the openings.

12. An adhesive member (10) according to any one of claim 1 wherein each opening (12) is positioned substantially diametrically opposite another opening (12).

13. An adhesive member (10) according to claim 3 wherein the opposing side walls (13, 14) of each opening (12) are moveable towards each other.

14. An adhesive member (10) according to claim 1 wherein the member (12) includes an aperture (16) in the central region thereof and wherein each opening (12) extends towards the aperture (16).

15. An adhesive member (10) according to claim 1 wherein one half of the member (10) has more of said openings (12) than the other half of the member (10).

16. An adhesive member (10) according to claim 1 wherein one half of the member (10) has three of said openings (12).

17. An adhesive member (10) according to claim 1 wherein upper one half of the member (10) has two of said openings (12).

18. An ostomy appliance (11) including an adhesive member (10) according to claim 1.

19. A one-piece ostomy appliance including an adhesive member (10) according to claim 1.

20. A two-piece ostomy appliance including an adhesive member (10) according to claim 1.
